(19) United States
(12) Patent Application Publication
(10) Pub. No.: US 2009/0298953 A1
(43) Pub. Date: Dec. 3, 2009

(54) Drug delivery system and method of making the same

(75) Inventors: Mohammad Imani, TEHRAN (IR); Hamid Mirzadeh, TEHRAN (IR)

Correspondence Address: BARRY CHOOBIN, TALEGHANI, BAHARE SHOMALI , #219, SUITE#18, TEHRAN, 1563714311, IR

(73) Assignee: Iran Polymer and Petrochemical Institute, Tehran (IR)

(21) Appl. No.: 12/499,867

(22) Filed: Jul. 9, 2009

(51) Int. Cl. A61K 47/30

(52) U.S. Cl. 514/772.3

(57) ABSTRACT

Disclosed is a method for making a composition which comprises of an array of unsaturated, aliphatic, biocompatible and biodegradable polyesters with photo curing capability which upon mixing with an active ingredient will provide an inject able liquid or putty-like material. The composition can be injected via a customary syringe and needle and be photocured in situ using visible light irradiation. Changing in the network crosslinking, molecular weight of the linear polymeric precursors and their chemical structures can control delivery rate of the active ingredient from the device.

### BACKGROUND OF THE INVENTION

In the recent years, extensive efforts are being made to develop novel ways for drug delivery to the affected tissues and body organs. To cause little tenacity and minimal side effects; It is necessary to deliver the biologically active agents to the target tissues in an optimal amount during the required period of time. In this regard, delivery of anti-cancer agents as inject able/implantable devices in cancerous tissues has attracted much attention to divert them away from the other organs and tissues in which toxicity arises especially considering the huge burden of side effects caused by these agents.

Biodegradable polymers play a major role as carriers in drug delivery systems since the mid-1970s. As these polymers degrade in the body to small molecular weight compounds that are either metabolized or excreted, they obviate the need for removal of the carrier after the device is exhausted.

Therefore, it would be advantageous to have an improved process if insertion.

Accordingly, the present invention discloses a method for fabricating an array of unsaturated, aliphatic, biocompatible and biodegradable polyesters with photo curing capability which upon mixing with an active ingredient will provide an inject able liquid or putty-like material. The composition can be injected via a customary syringe and needle and be photo cured in situ using visible light irradiation. Changing in the network cross-linking, molecular weight of the linear polymeric precursors and their chemical structures can control delivery rate of the active ingredient from the device.

### FIELD OF THE INVENTION

The present invention relates to the synthesis of novel unsaturated polyester macromers, which are clear in color, biodegradable, biocompatible and tunable in properties. The prepared compositions based on these materials can be used as injectable drug delivery devices upon compounding, injection to the desired site and visible light irradiation.

### BACKGROUND OF THE ART

Using in situ forming devices as injectable/implantable drug delivery systems has attracted much attention due to several potential advantages over conventional prefabricated ones. These advantages include the possibility of using the initial materials as liquids or moldable putties and forming them easily in complex shapes which upon the subsequent reaction, a solid implant of exactly the required dimensions will be shaped without need to any invasive surgical process for implantation. Some reactions may be used to transform a liquid polymeric sol to a solid/semisolid gel including application of thermoplastic pastes, polymer precipitation from solution, ion-mediated gelation or chemical cross-linking. Administration of these devices via injection through a customary syringe and needle will also reduce the invasiveness of insertion process of traditional implants. Using biodegradable and biocompatible polymers in this construct; there will be no need for a removal procedure to be performed surgically after the effective period of drug delivery. Finally, localized or systemic drug delivery can be achieved for prolonged periods of time, typically ranging from one to several months.

Thermoplastic pastes are polymeric compositions, which their melt are injected into the body and form a semi-solid depot upon cooling to body temperature. The polymers used in this way should melt at temperatures near to the physiologic one i.e. 37° C. also low melt viscosity so, flow easily when pushed or stretched by a load, usually at elevated temperatures. Polymers of low molecular weights and glass transition temperatures are the best candidates to meet these requirements and provide a facile inject ability however; melting points ranging from 25 to 65° C., and intrinsic viscosities of 0.05 to 0.8 dl/g (at 25° C.) are reported for these devices which are far from the prerequisites hence, high temperature at the time of injection will be observed.

Polymer precipitation from solution is also reported to produce an injectable drug delivery depot. Precipitation can be induced by solvent-removal; a change in the temperature or pH, which can be described as solvent-removal precipitation and thermally, induced sol-gel transition, respectively. These devices are comprised of, for example; a water insoluble biodegradable polymer dissolved in a water miscible, physiologically compatible solvent. Upon injection into the aqueous environment of human body, the solvent diffuses into the surrounding aqueous environment while water diffuses into the polymer matrix. Since the polymer is water insoluble, it precipitates upon contact with the water and results in a solid polymeric implant. The most critical property for a solvent to be used in this system is their capabilities to form hydrogen bonding with water. Many drug candidates have been examined using this system including gonadotrophins, Chlorhexidine, doxycycline, naproxen and theophylline. Although sustained release was achieved and some commercial products were introduced in the market in this way but high initial burst release, relatively rapid release rates, and use of controversial solvents are the case with them yet. Many polymers including acrylamide-based ones and amphiphilic copolymers also undergo abrupt changes in solubility in response to changes in the environmental temperature. This physical characteristic has been employed to form drug depots by using polymer systems, which undergo a sol-gel transition upon injection into the body. Acrylamide-based polymers are not generally suitable for injectable/implantable devices due to their inherent toxicity

Organogels are composed of water-insoluble amphiphilic lipids, which swell in water and form various types of lyotropic liquid crystals. The nature of the liquid crystalline phase formed depends on the structural properties of the lipid, temperature, nature of the drug incorporated, and the amount of water in the system. They are promising injectable delivery systems for lipophilic compounds. Stability of oils and purity of waxes which are used in their composition along with very broad phase transition with temperature are the common problems in this way. Lacks of toxicity data and phase separation that can easily reduce the potency of some drugs are common problems.

Unfortunately, most of the methodologies which have been used to provide such devices achieved limited success to control the gelation kinetics and hence the properties of the resulting materials. However, strict controlling of the crosslink density in the strategies based on free radical reaction (initiated by heat, redox, or photo irradiation mechanisms) will provide the readily adjustable network properties including permeability, degradation, water uptake, or mechanical properties.

Thermoses macromers can flow and be molded when initially constituted hence will provide the required flow for injectability. Soon after crosslinking reaction initiated by heat or redox initiation systems, they will irreversibly turn to solid bodies. In the most cases, crosslinking reaction means the formation of covalent bonds between neighboring polymer chains, which ends to a macromolecular network. Irreversibility of these bonds provides long life to the device i.e. high thermal and mechanical stability. The advantage of using this system is its facile syringeability. And there are some disadvantages like unacceptable level of heat released during the reaction, and burst in drug release and toxicity of un-reacted monomers.

Photocrosslinked gels are another type of in situ crosslinked systems who provide many advantages over chemically initiated thermoset systems. These include rapid polymerization rates in physiological temperatures, achieving complex shaped with exactly the required dimensions. In this approach, prepolymers, mostly based on polyanhydrides are introduced to the desired site via simple injection and photocured in situ with fiber optic cables. Numerous medical applications may benefit from such compositions e.g. in dentistry ceramic filled dimethacrylate monomers are photo polymerized with blue light to produce tooth colored restorations in situ as an alternative to mercury amalgam fillings. Some prior art presented some of the first works using degradable polymers that were photocured in vivo to prevent post-operative adhesions. Second, the adhesion of the polymer to surrounding tissue is generally significantly improved because of intimate contact of the polymer with the tissue during formation and the resulting mechanical interlocking that can arise from surface microroughness. Third, the invasiveness of some surgical techniques is minimized as liquid solutions are easily introduced through needle injections and can be photocured with fiber optic cables using arthroscopic techniques.

Most of the reports on photo crosslinkable macromers are devoted to acrylate-based monomers, which are generally well recognized as unsafe materials. There are few unsaturated moieties available as alternatives to acrylate-based monomers such as fumarate and itaconate monomers. Some macromers reported based on fumarate macromers are polyethylene glycol fumarate, polypropylene fumarate and poly(ε-caprolactone fumarate). Degradation products resulted from these copolyesters is completely biocompatible and will be metabolized in Krebs's cycle. The photoinitiated crosslinking of unsaturated macromers by visible light to produce three-dimensional polymeric networks seems interesting due to its rapid and effective nature. In this way, the initiation time of the reaction will be easily controlled and can be carried out at lower temperatures with minimal heat generation. Using visible light in the photocuring of transparent, unsaturated, and liquid or putty-like materials, thick layers (in much more depth) could be cured rapidly due to tendency of the corresponding photoinitiators, for example, camphorquinone, to quickly photobleach. This is why this method has found more versatile applications than UV or gamma ray curing. In this way, due to the very mild reaction conditions, the polymerization can be carried out in direct contact with drugs, cells, and tissues.

In addition to the advantages mentioned before, these systems have some problems like shrinkage and brittleness of the polymer due to high degree of crosslinking.

One of the major problems facing cancer chemotherapy is the achievement of the required therapeutic concentration of the drug at the tumor site for a desired period of time without causing undesirables effects on the organs while circulating in the body. The vascular system of tumors is highly disorganized and unpredictable both in its structure and in function. This disorganization serves as a major barrier in the delivery of drugs to solid tumors. High viscosity of blood in the tumor significantly hinders drug delivery to poorly perfused regions of tumor mass. Another factor that poses a problem to drug delivery in solid tumors is the abnormally high pressure in the interstitial matrix of the tumor that retards the passage of molecules across the vessel walls and into the interstitial matrix. Oral administration of the non-steroidal anti-estrogen like Tamoxifen is the treatment of choice for the patients with all stages of estrogen receptor (ER) positive breast cancer. Despite being quite effective, tamoxifen can have harmful long term side effects such as the development of endometrial cancer, or an acquired Tamoxifen resistance leading to further tumor progression. To overcome these undesirable side effects, one could encapsulate Tamoxifen in stealth PEGylated nanoparticles.

Microparticulate drug delivery systems are considered and accepted as a reliable means to deliver the drug to the target site with specificity, if modified, and to maintain the desired concentration at the site of interest without outward effects. Sehra et al. prepared biodegradable microspheres of PLGA 65:35 by o/w emulsification solvent evaporation method, and they synthesized different batches of varying concentration of drug, polymer, polyvinyl alcohol and solvent. They demonstrated that concentration of polymer, drug and stabilizer affects the part size, encapsulation efficiency and drug release rate.

Oral administration of the non-steroidal anti-estrogen like Tamoxifen is the treatment of choice for the patients with all stages of estrogen receptor (ER) positive breast cancer. Tamoxifen citrate, a non steroidal antiestrogen has potential applications in treatment of breast cancer. This drug is slightly soluble in water.

Tamoxifen, an antiestrogen, is widely used as an adjuvant in the treatment of breast cancer. Tamoxifen has undergone clinical trials in Europe, as well as in the United State and Canada, to evaluate its preventive effect on breast cancer in women at high risk. Recently, TAM has been used to treat other cancers, such as liver, brain and pancreas. It has been reported that TAM and its active metabolite 4-hydroxytamoxifen exert anti-oxidative effects in vitro.

### SUMMARY OF THE INVENTION

The principal object of the present invention is to provide a method for controlled delivery of a predetermined amount of a drug wherein said method comprises;

Combining a macromer with a predetermined amount of a drug, and a plurality of initiators, and obtaining a first composition,

Applying a crosslinking procedure to said first composition, wherein said drug is unaffected by said crosslinking; and

Obtaining a second composition, wherein said second composition controls said delivery of said predetermined amount of said drug.

Yet another object of the present invention is to provide macromer which comprises of unsaturated aliphatic polyesters, wherein said unsaturated aliphatic polyesters are a result of a polyesterification reaction catalyzed by a catalyst between a diol, wherein said diol comprises polyethyelene glycol, polycaprolactone diol, and polyhexamethylene carbonate diol and an acyl halide wherein said acyl halide comprises of fumaryl chloride and itaconyl chloride.

Yet another object of the present invention is to provide catalyst, which comprises of epoxy containing compounds wherein said compounds comprise propylene oxide, and 1,2-butylene oxide.

Yet another object of the present invention is to provide macromer, which is a white clear macromer.

Yet another object of the present invention is to provide macromer which is crosslinked by visible light initiation, wherein said visible light initiation characterized with duration and intensity of said visible light irradiation.

Yet another object of the present invention is to provide macromer which is crosslinked by thermal initiation, wherein said thermal initiation characterized with duration of crosslinking, temperature of crosslinking and concentration of said thermal initiation.

Yet another object of the present invention is to provide macromer which is crosslinked by redox initiation, wherein said redox initiation characterized with duration of crosslinking, and concentration of said redox initiation.

Yet another object of the present invention is to provide a method in which said controlled delivery of said predetermined amount of said drug is based on intensity and duration of said visible light irradiation.

Yet another object of the present invention is to provide a method in which said controlled delivery of said predetermined amount of said drug is based on said duration of crosslinking, said temperature of crosslinking and said concentration of said thermal initiation.

Yet another object of the present invention is to provide a method in which said controlled delivery of said predetermined amount of said drug is based on said duration of crosslinking, and said concentration of said redox initiation.

Yet another object of the present invention is to provide a composition for controlling duration and amount of delivery of a predetermined amount of a drug, wherein said composition comprises:

A first composition comprising a macromer, a predetermined amount of a drug, and a plurality of initiators, wherein said macromer is subject to a crosslinking procedure, thereby obtaining a second composition, wherein said second composition controls said duration and amount of delivery of said predetermined amount of said drug.

Yet another object of the present invention is to provide a composition for controlling duration and amount of delivery of a predetermined amount of a drug wherein said macromer comprises of unsaturated aliphatic polyesters, wherein said unsaturated aliphatic polyesters are a result of a polyesterification reaction catalyzed by a catalyst between a diol, wherein said diol comprises polyethylene glycol, polycaprolactone diol, and polyhexamethylene carbonate diol and an acyl halide wherein said acyl halide comprises of fumaryl chloride and itaconyl chloride.

Yet another object of the present invention is to provide a composition in which catalyst comprises of epoxy containing compounds wherein said compounds comprises propylene oxide, and 1,2-butylene oxide.

Yet another object of the present invention is to provide a composition in which macromer is a white clear macromer.

Yet another object of the present invention is to provide a composition in which macromer is crosslinked by visible light initiation, wherein said visible light initiation characterized with duration and intensity of said visible light irradiation.

Yet another object of the present invention is to provide a composition in which macromer is crosslinked by thermal initiation, wherein said thermal initiation characterized with duration of crosslinking, temperature of crosslinking and concentration of said thermal initiation.

Yet another object of the present invention is to provide a composition in which macromer is crosslinked by redox initiation, wherein said redox initiation characterized with duration of crosslinking, and concentration of said redox initiation.

Yet another object of the present invention is to provide a composition in which controlled delivery of said predetermined amount of said drug is based on intensity and duration of said visible light irradiation.

Yet another object of the present invention is to provide a composition in which controlled delivery of said predetermined amount of said drug is based on said duration of crosslinking, said temperature of crosslinking and said concentration of said thermal initiation.

Yet another object of the present invention is to provide a composition in which controlled delivery of said predetermined amount of said drug is based on said duration of crosslinking, and said concentration of said redox initiation.

This present invention comprises synthesis of biodegradable, biocompatible, unsaturated polyesters based on different diols including polyethylene glycol, poly (ε-caprolactone diol) and poly(hexamethylene carbonate diol) and diacids comprising fumaric acid and itaconic acid. Polyesters were synthesized via reaction of precursor diol with acyl halide derivative of diacid in the presence of propylene oxide as a catalyst and proton scavenger. The resulting polymers were completely white clear materials suitable for photo crosslinking.

Injectable polymeric compositions based on an unsaturated polyester e.g. poly (ethylene glycol) fumarate (PEGF), poly (ε-caprolactone fumarate), poly (hexamethylene carbonate fumarate) or their corresponding itaconate derivatives were crosslinked using N-vinyl pyrrolidone (NVP) as a reactive diluent also crosslinking agent, N, N dimethyl p-toluidine (DMPT) as an accelerator and a photo initiator such as comphorquinone (CQ). Samples were photo cured after adding the corresponding amount of active ingredient i.e. tamoxifen citrate to the above composition.

The present invention includes a new method in which a macromer that is injectable, biodegradable can be in situ photo crosslinked by using visible light source and makes networks, which are useful for biomedical application. To achieve these objectives, optically transparent and biodegradable macromers based on polyethylene glycol, poly (ε-caprolactone diol) and poly (hexamethylene carbonate diol), and diacids comprising fumaric acid and itaconic acid were synthesized using propylene oxide as a different proton scavenger to enhance in situ photocrosslinking capability. The macromers in different compositions were then photocrosslinked for 300 sec in the presence of a visible light initiator/accelerator couple and also a reactive diluent.

In this invention includes some methods in order to characterize crosslinked networks, there are applied some studies e.g., determination of the degree of conversion, measurement of the shrinkage strain and initial shrinkage strain rates and equilibrium swelling and sol fraction study and also some dynamic mechanical analysis of the resulting networks. The macromer may also be advantageous for in situ visible photo curing because they are colorless. This reaction may be useful for other applications. Further, the reaction can be influenced by a factor such as diol molecular weight. Fabrication of networks using visible light photo crosslinking is also described. Application of these networks in drug delivery was also described.

### BRIEF DESCRIPTION OF DRAWINGS

FIG. 1 is a schematic representation of macromer synthesis pathway and subsequent photo crosslinking in the presence of NVP for PEGF as an example;

FIG. 2 shows FTIR spectra of PEG 1 kDa and PEGF obtained from different initial molecular weights of precursor diols;

FIG. 3 shows 1HNMR spectra of the PEG 1 kDa, PEGF 0.4 and 1 kDa in CDCl3;

FIG. 4 shows Shrinkage strain of PEGF 1 kDa based specimens with different contents of NVP during photo crosslinking;

FIG. 5 shows Shrinkage strain of PEGF 0.4 kDa based specimens with different contents of NVP during photo crosslinking;

FIG. 6 shows comparison of the shrinkage strain rates of PEGF 0.4 kDa with different contents of NVP during photo crosslinking;

FIG. 7 shows correlation between shrinkage strain and NVP-fraction of the hydrogels (n=3);

FIG. 8 shows degree of conversion for PEGF 0.4 and 1 kDa based formulations with different NVP contents (n=3);

FIG. 9 shows NVP content dependence of the Mc values for PEGF 0.4 and 1 kDa swollen in water (20° C.);

FIG. 10 shows cell Culture test, L929 fibroblast cells growing neighboring to crosslinked networks, (X=400); and

FIGS. 11A-11G illustrate in vitro release profiles of TMX released from different formulations of PEGF 0.4 KDa (n=4). L stands for drug loading (% w/w) and D stands for disk thickness (mm).

### DETAILED DESCRIPTION OF THE INVENTION

### Materials and Methods

Materials

Polyethylene glycol (Mw=0.4 and 1 kDa), poly (ε-caprolactone diol) (Mw=0.63, 1.2 and 2 kDa) and poly (hexamethylene carbonate diol) (Mw=0.83 and 2 kDa), N-vinyl-2-pyrrolidone (NVP), camphorquinone (CQ), calcium hydride, fumaryl chloride (FuCl), itaconyl chloride (ItCl) and propylene oxide (PO) were all purchased from Aldrich (Milwaukee, Minn., USA). N, N-Dimethyl-p-toluidine (DMPT), sodium hydroxide (NaOH) and methylene chloride (DCM) were obtained from Merck (Germany). FuCl was purified by distillation at 161° C. under ambient pressure. Anhydrous DCM was obtained by distillation under reflux condition for 1 hour in the presence of calcium hydride. NVP was also distilled under reduced pressure (30 mmHg). Tamoxifen citrate (TMX) was a kind gift from Iran Hormon Co. (Iran). Other solvents and reagents were of the reagent grade and used without further purification.

Synthesis of Macromers

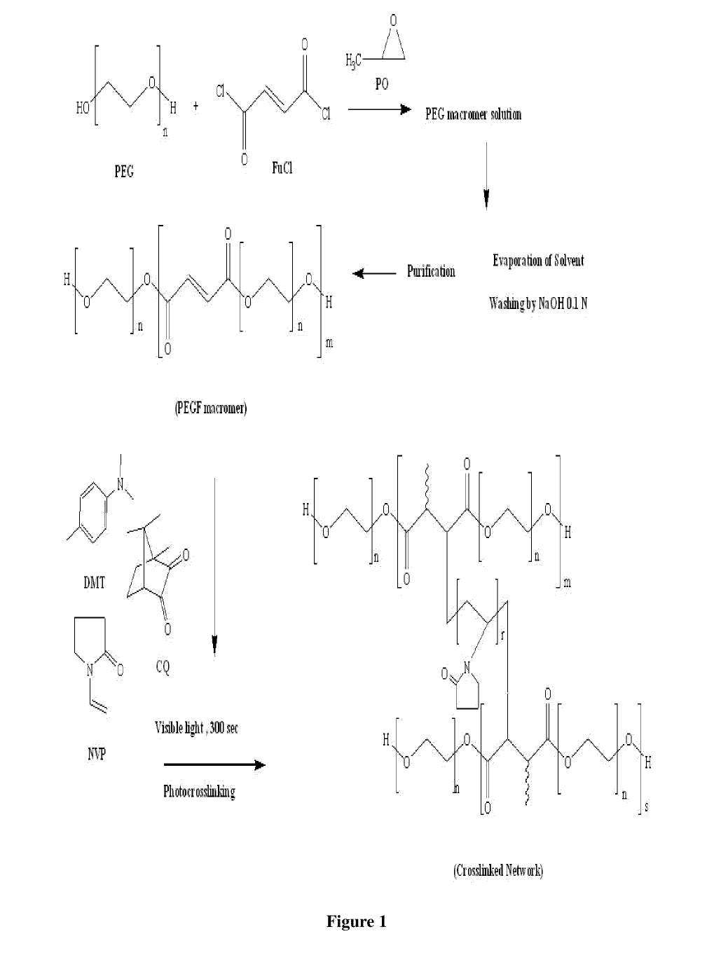

As shown in FIG. 1 for PEGF macromers (as an example) were synthesized according to the procedure which is depicted in FIG. 1. Typically, 0.03 mole of diol i.e. PEG diol was dissolved in 100 mL of anhydrous DCM in a three necked 250 mL reaction flask equipped with reflux condenser and magnetic stirrer. PO was added to the mixture in a 2:1 molar ratio. The purified diacid i.e. FuCl or ItCl (0.995:1 molar ratio to diol) was dissolved in 50 mL of the same solvent and added dropwise in one hour to the stirred reaction flask at −2° C. under nitrogen atmosphere. The reaction temperature was then raised to the room temperature and run overnight. Upon completion of the reaction, the product was washed several times with 0.1N sodium hydroxide (NaOH) to remove the resulted byproducts such as chlorinated propanols. The macromer was then obtained by rotovaporation, dried at 25° C. in vacuum for 24 hours. Then, which was reserved at −15° C. until further applications.

Photo Polymerization of Macromers

Macromers were crosslinked by visible light in the presence of CQ and DMPT as photo initiator system (FIG. 1). Typically, 0.3 mg of macromer was dissolved in 1 μL of DMPT (26 mg) containing NVP and the equivalent of CQ (1:1 w/w to DMPT) was mixed thoroughly in this solution according to a 2 level factorial design. The factors were comprised of the initial diol molecular weights (e.g. 0.4 & 1 kDa for PEG diol) and the reactive diluent percentage (8 & 12%). The reported measurements for the experimental design are represented as means±standard deviation. The mixture was then cast in molds with a definite geometry (cylindrical shape with 8 mm diameter and 1 mm height) and cured for 300 seconds using a blue light source with an irradiance of circa 450 mW/cm2 (Optilux 501, USA) to prepare the specimens.

Macromers Characterizations

FTIR spectra (4000-400 cm-1) were obtained on a Bruker, Equinox 55 spectrophotometer at 4 cm-1 resolution and 32 scans. The specimens were analyzed on KBr disks at room temperature. 1HNMR spectra were recorded in CDCl3 at 25° C. (Bruker Ultrashield® 400 MHz, Germany) and chemical shifts were recorded in ppm.

Gel permeation chromatography (GPC) was accomplished using a GPC instrument (Shimadzu, CR4AX, GC15A, Japan). Polystyrene of known molecular weights were used as the calibration standards. THF was used as the mobile phase eluting at a flow rate of 1.0 mL/min. A 100 μL sample of 0.1 mg/mL solution of the macromer in THF, which was filtered through a 0.22 μm filter prior to use, was injected for all measurements.

Thermal Analysis

Melting point (Tm), glass transition temperature (Tg) and crystallinity of the samples were evaluated using a TA instrument 920 differential scanning calorimeter (DSC) under nitrogen gas flow rate of 100 mL/min measured at a heating rate of 10° C./min via heating from −80° C. to 100° C. First, the specimens were heated from −80 to 100° C. at heating rate of 10° C./min, and then quenched rapidly to −80° C. The glass transition temperature, Tg, was taken as a midpoint of the heat capacity change. Tm and heat of fusion (ΔHm) were determined from the maximum endothermic peaks position and integrating of endothermic area.

Polymerization Shrinkage Strain and Shrinkage Strain Rate Measurement

The bounded disk technique was used to measure the shrinkage of light cured samples. Briefly, the specimen to be cured was placed at the center of a brass ring (with square cross section) adhesively bonded to a rigid glass plate and the top edge of the ring and the disk specimen were covered by a flexible diaphragm e.g. a microscope lamella. A centrally aligned LVDT displacement transducer was positioned in contact with the center of the cover slip. The light source was beneath the rigid glass plate and upon initiation of reaction the cover slip deflected due to polymerization shrinkage and LVDT transducer which was connected to the signal conditioning unit, microcomputer transient recorder and data logging system, monitored the deflection of cover slip over time. The total shrinkage strain of the sample was assessed for 400 seconds after starting the light irradiation, at which time the contraction had plateau-out.

Measurement of Conversion

To determine the degree of conversion (DC), the specimens were placed between two polyethylene films and pressed to form a very thin film. FTIR absorbance spectra of the samples were recorded before and after curing reaction. DC % was determined from the ratio of absorbance intensities of aliphatic C═C (peak at 1645 cm-1) against the internal references (peaks at 724, 1463 cm-1) before and after curing of the specimen. The degree of conversion was then calculated as follows (eq. (1)):

$$DC \% = ( 1 - \frac{\begin{matrix}( \frac{1645 {cm}^{- 1}}{Reference Peak {cm}^{- 1}} ) \\ peak area after curing\end{matrix}}{\begin{matrix}( \frac{1645 {cm}^{- 1}}{Reference Peak {cm}^{- 1}} ) \\ peak area before curing\end{matrix}} ) \times 100 \qquad ( 1 )$$

Equilibrium Swelling and Sol Fraction Study

The equilibrium swelling of the photocrosslinked networks was investigated by a gravimetric method. All of samples were molded as previously described and weighed in dry state, Wi. Then the disks were placed in 50 mL of deionized distilled water (DDW) until equilibrium and weighed again, Ws. The swollen gels were dried overnight at reduced pressure, and then weighed, Wd. Swelling data were used to calculate the equilibrium swelling ratio and sol fraction percent for each formulation using the following formulas (eqs. (2,3):

$$\begin{matrix}Ratio = \frac{{W}_{s} - {W}_{d}}{{W}_{d}} & ( 2 ) \\ Sol fraction \% = \frac{{W}_{i} - {W}_{d}}{{W}_{i}} \times 100 & ( 3 )\end{matrix}$$

DMA

The rheological measurements were carried out using a Paar-Physica oscillatory rheometer (MCR300, Germany) at 20° C. with parallel plate geometry (plate diameter of 8 mm, gap of 1-1.5 mm). The tests were accomplished at 20° C. in order to avoid water evaporation during the experiment. The strains used were chosen to be in the linear viscoelastic (LVE) range, where G′ and G″ are independent of the strain amplitude. LVE range was determined for the photo crosslinked gels in the swollen state in water. The specimen was placed between the parallel plates of the rheometer and a strain sweep test (ω=1 rad/s) was conducted. The test conditions for the frequency sweeps were selected to confirm that the test is really carried out in the LVE range (shear strain of 0.2%). Then a frequency sweep was performed and a graph of G′ and G″ versus frequency were achieved in a range of 0.1-100 Hz at constant temperature at 20° C.

Biocompatibility Assay

Cell culture was performed on photocrosslinked samples using L929 fibroblast cells of mice as a test model. The cells were maintained in growth medium RPMI-1640 supplemented with 100 IU/mL of penicillin, 100 μg/mL of streptomycin and 10% fetal calf serum. A routine subculture was used to maintain the cell line. The cells were incubated in a humidified atmosphere of 5% CO2 at 37° C. After one week incubation the monolayer was harvested by tripsinization. The samples were sterilized in an autoclave and placed in a multiple tissue culture polystyrene plate with 5 mL of cell suspension and then maintained in incubator for 48 hours. One sample was kept as a negative control. After incubation the samples were taken away from the incubator and examined for morphology and cell growth.

In vitro Drug Release

The resulting macromer was dried at 25° C. in vacuum oven for 24 hours and stored at −15° C. until further use. macromer was crosslinked by visible light in the presence of CQ and DMPT as photo initiator system. The CQ/amine photo initiator system for generation of radicals is widely used for the curing of dental restoration materials. First, Appropriate amount of the macromer was mixed with TMX (1 or 2% wt % macromer as drug loading) so the homogeneous material was obtained and then DMT and the corresponding amount of CQ (1:1 w/w to DMT) dissolved in NVP (12% wt total) and was added to the macromer mixture. The specimens (cylindrical shape with 8 mm diameter and 1 or 2 or 3 mm height) were cured for 300 seconds using a blue light source with intensity of 450 mW/cm2 (Optilux 501, USA).

The release profile of TMX from different geometries with different loading were determined in 10 mL PBS solution pH 7.4 containing 30% v/v Isopropanol providing sink conditions in a thermostatic bath system at 37° C. Samples were withdrawn at given time intervals and replaced with fresh buffer solution maintained at the same temperature. Samples was then analyzed for TMX concentration with UV-VIS spectrophotometer (UV1650PC, Shimadzu, Japan) using an analytically validated method (r2>>0.99) at 277 nm.

### Results

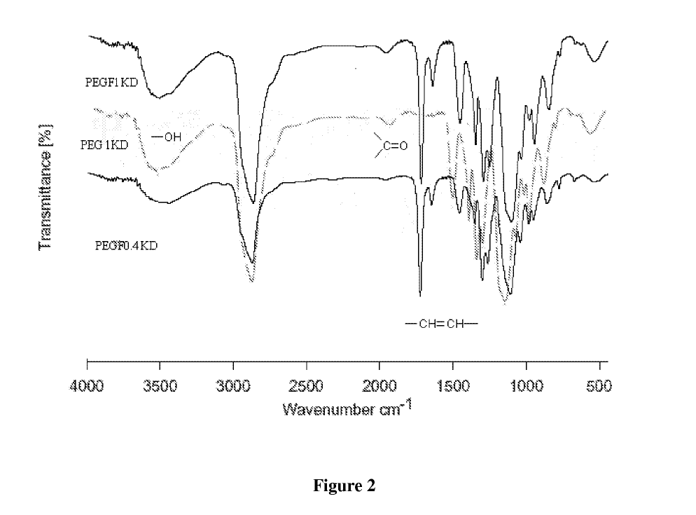

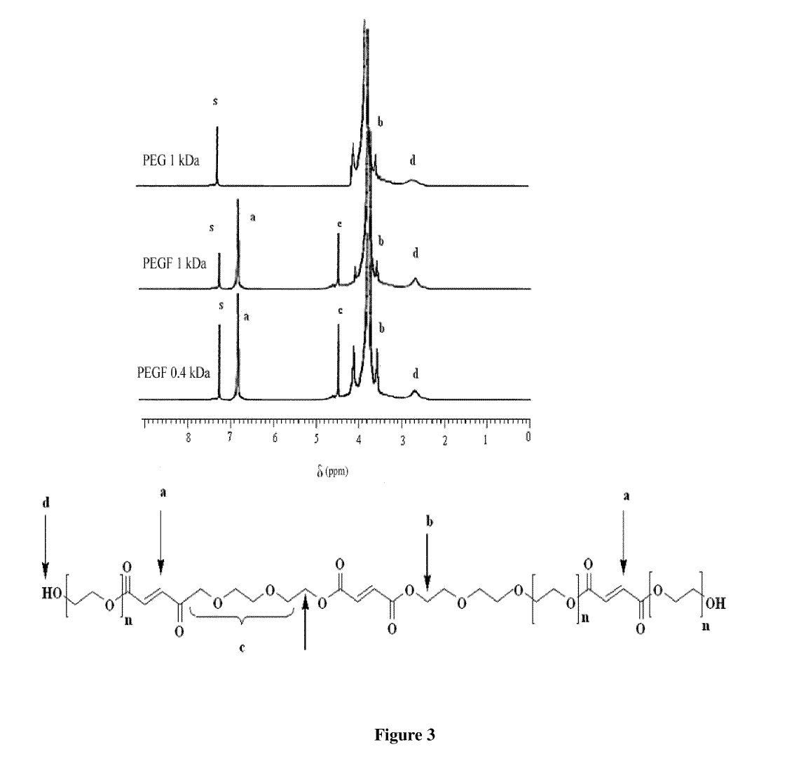

As shown in FIG. 2, the FTIR spectra of PEGF 0.4 and 1 KDa are presented as an example. Asymmetrical C-O-C stretching band at 1100 cm⁻¹, C═C stretching at 1645 cm⁻¹, carbonyl stretching at 1720 cm-1, strong methylene absorption at 2871 cm-1, methylene scissoring and asymmetric bending at 1455 cm-1 and hydroxyl absorption at 3442 cm-1 are evident and can be found. The absorption bands presented at 950 and 858 cm-1 positioned in the FTIR spectra are characteristic of the crystalline phase of PEG.

As shown in FIG. 2, 1HNMR spectra of the synthesized PEGF macromers are shown as an example. The chemical shifts with peak positions at 3.63, 4.33, 2.7 and 6.8 ppm are due to the protons of PEG main ethylene (b), methylene groups adjacent to the fumarate groups (c), the hydroxyl group of PEG (d) and hydrogens of the fumarate group (a), respectively. Since the chemical shift of the fumarate hydrogens is below 7.0 ppm, the steric configuration of the fumarate functional groups in the copolymer should be in the cis position. The presence of chemical shift at 6.8 ppm clearly reveals that fumarate groups are incorporated in PEG.

According to the GPC results; no significant effect on number-average molecular weight (Mn) of the resulted PEGF macromers were observed with increasing PEG diol molecular weights (from 0.4 to 1 kDa). This is in contrast to what observed with weight-average molecular weight (Mw) of the macromers which implies a larger polydispersity index (PDI). Table 1 indicates Mn, Mw and PDI for these macromers. In comparison with PEG diol of 1 kDa, 0.4 kDa, PEG diols of 0.4 kDa have more reactive hydroxyl end groups since the larger PEG random coil exerts more steric hindrance, so Mn and Mw of PEGF 0.4 kDa increase considerably. For that reason, our results clearly prove that the hydroxyl groups of low molecular weight PEGs are also more accessible to fumaryl chloride during the oligomerization (FIG. 2 and Table 1).

TABLE 1

| Sample | Mn | Mw | PDI |
|---|---|---|---|
| PEG 0.4 kDa | 380 | 420 | 1.10 |
| PEGF 0.4 kDa | 3050 | 5800 | 1.90 |
| PEG 1 kDa | 930 | 1190 | 1.27 |
| PEGF 1 kDa | 3440 | 8380 | 2.43 |

Crosslinking characteristics in terms of maximum shrinkage strain (and strain rate), ultimate shrinkage strain and time at maximum shrinkage strain rate are reported in Table 2. Maximal shrinkage strain for PEGF 1 kDa samples are lower than the corresponding 0.4 kDa samples due to the fewer number of double bonds (nearly 3 times), and hence the ultimate shrinkage strain will be smaller in turn.

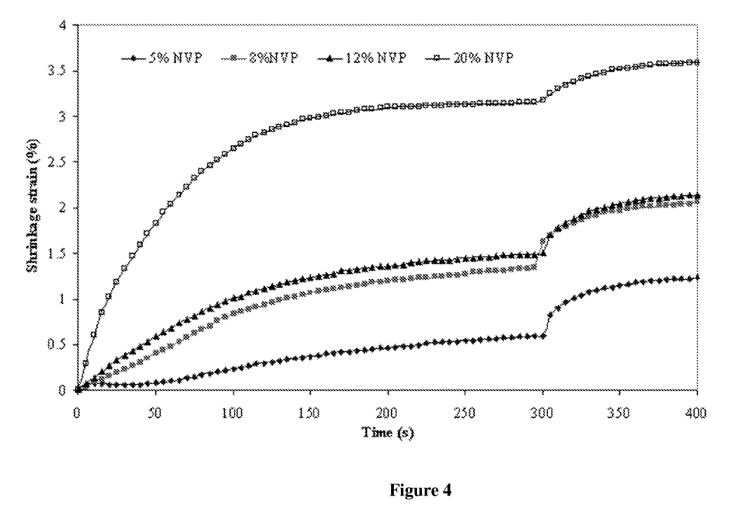

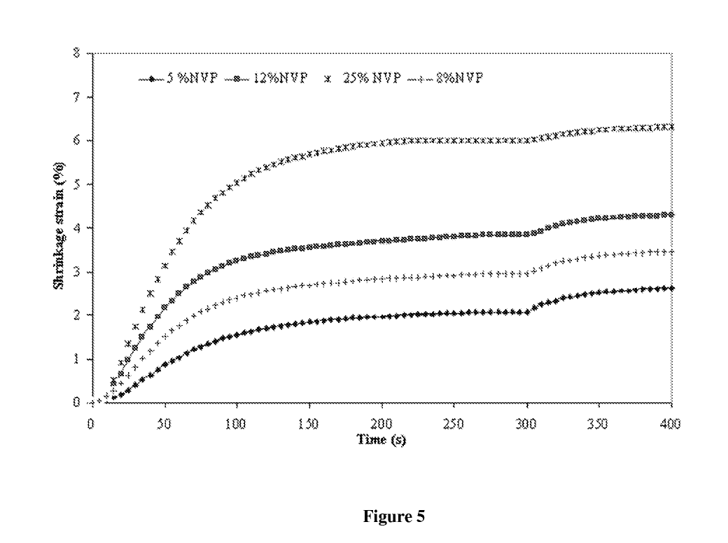

FIGS. 4 and 5 show the shrinkage strains of PEGFs 0.4 and 1 kDa with different NVP contents. The results indicate that the total shrinkage strain was increased with increasing NVP content due to further conversion of weak intramolecular Van der Waals forces to the strong covalent single bonds during the crosslinking reaction. By increasing in molecular weight of the PEGF macromers or decreasing NVP content, viscosity of the mixture is also increased which in turns affects the photocrosslinking reaction via limiting the diffusion of free radicals. This phenomenon would decrease the maximum observed shrinkage strain rate and increase the time at which maximum shrinkage strain rate are observed at the set point of the mixture. As the viscosity increases, the limited diffusion of radicals may interfere with the termination step of the crosslinking reaction by bimolecular coupling. Therefore, this may be another reason for low shrinkage strain observed in PEGF 1 kDa specimens (FIG. 5).

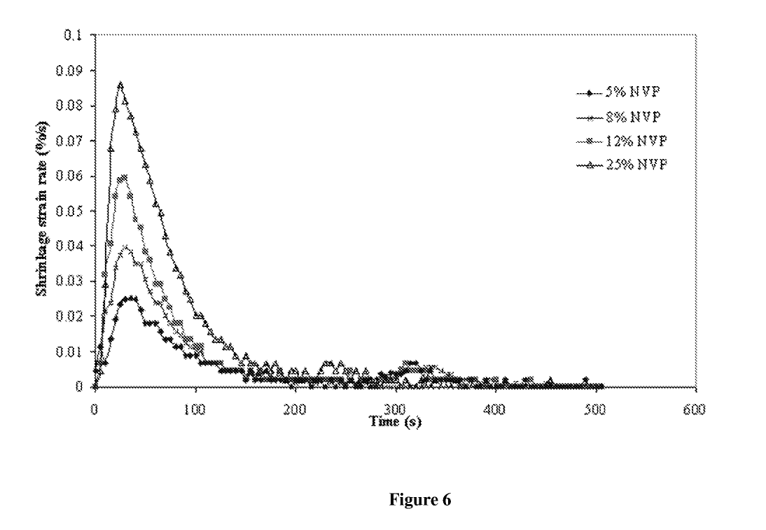

As shown in FIG. 6, as the shrinkage is the consequence of the polymerization reactions, it should follow the polymerization reaction model. Shrinkage strain rate, which is related to polymerization rate, is an important factor affecting the biomechanics and marginal integrity of the crosslinked polymer cured to form a biomedical device. FIG. 6 depicts the shrinkage strain rate behavior of PEGFs 0.4 kDa which shows that the ultimate shrinkage strain rate being dependent on the NVP content. The time at which the maximum shrinkage strain rate is reached is also shown by tεm. As illustrated in Table 2, there is a significant difference between the tεm of PEGFs 0.4 and 1 kDa with different NVP contents. Increasing in the NVP content will decrease the time at which the shrinkage strain rate reaches its maximum due to the more accessible crosslinking agent also lower viscosity of the mixture. As a consequence, the species with more NVP content have higher shrinkage strain rates and faster crosslinking reactions at the early times.

TABLE 2

| | Max. | Ultimate | Max. | Time at |
|---|---|---|---|---|
| | shrinkage | shrinkage | shrinkage strain | max. shrinkage |
| Samples | strain (%) | strain (%) | rate (%/s) | strain rate (s) |
| PEGF 0.4 | 2.06 ± 0.01 | 2.64 | 0.025 ± 0.001 | 40 ± 6.50 |
| kDa - 5% NVP | | | | |
| PEGF 0.4 | 2.95 ± 0.07 | 3.45 | 0.038 ± 0.004 | 35 ± 2.50 |
| kDa - 8% NVP | | | | |
| PEGF 0.4 | 3.92 ± 0.06 | 4.32 | 0.058 ± 0.003 | 30 ± 3.10 |
| kDa - 12% NVP | | | | |
| PEGF 0.4 | 6.14 ± 0.11 | 6.37 | 0.085 ± 0.006 | 25 ± 1.90 |
| kDa - 25% NVP | | | | |
| PEGF 1 | 0.72 ± 0.001 | 1.24 | 0.01 ± 0.00 | 80 ± 6.70 |
| kDa - 5% NVP | | | | |
| PEGF 1 | 1.45 ± 0.06 | 2.05 | 0.09 ± 0.00 | 50 ± 5.20 |
| kDa - 8% NVP | | | | |
| PEGF 1 | 1.75 ± 0.08 | 2.14 | 0.13 ± 0.00 | 40 ± 3.20 |
| kDa - 12% NVP | | | | |

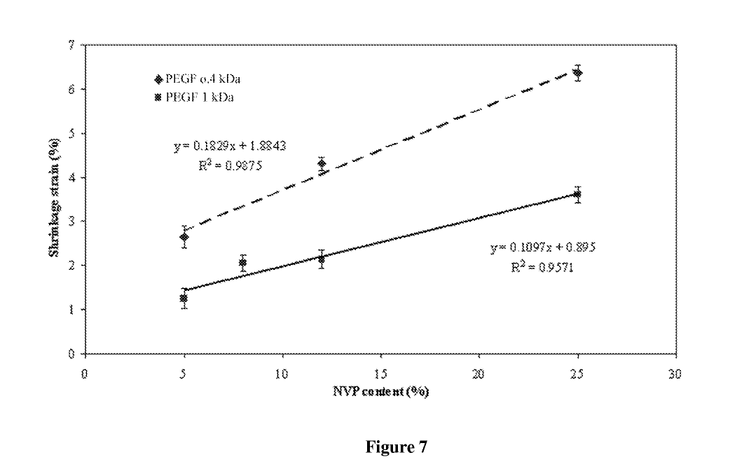

FIG. 7 denoted a linear correlation (R2>>0.95) between NVP content and shrinkage strain. The slope of shrinkage strain of PEGF 0.4 kDa and the intercept of linear plot of PEGF 0.4 kDa are more than PEGF 1 kDa. The shrinkage strain percentages are increased more in PEGF 0.4 kDa upon light irradiation due to the higher amount of double bonds present also its lower viscosity in comparison to the PEGF 1 kDa. This will cause NVP molecules to be more mobile and place speedily among the chains to make a crosslinked networks.

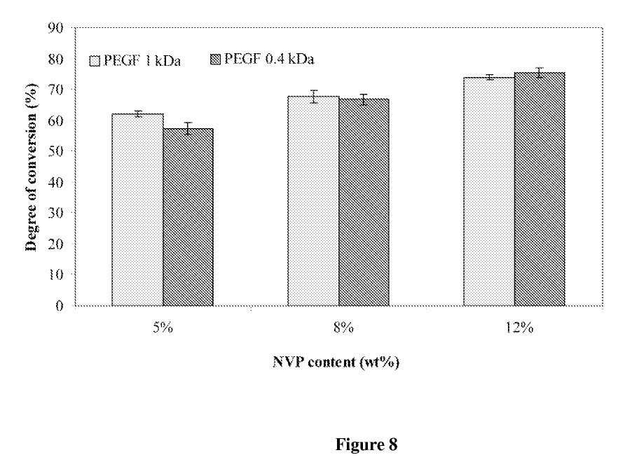

The increase in molecular weight of PEG increases viscosity of PEGFs as well as decreases the reactivity. The time and the conversion at which the maximum strain rate was acquired decreased with an increase in the molecular weight of PEGFs. The very high viscosity of PEGF hinders the mobility of growing macroradicals and monomers and causes the maximum rate appearing at longer times. However, there is some increase in PEGFs viscosities due to increasing in PEGs molecular weights but the number of double bonds strongly influences the curing conversion of PEGF 0.4 and 1 kDa in contrast to NVP content (FIGS. 6, 8).

As mentioned before, the increase in NVP content results in an increasing in degree of conversion which means enough double bonds for NVP as a crosslinker to be placed among them and result in the corresponding network. As shown in FIG. 8 the decrease in conversion of PEGF 0.4 kDa and PEGF 1 kDa with the same NVP contents shows no considerable changes in 8% and 12% NVP content (p<0.05). The outcomes of the amount of double bonds also viscosities which are related to PEGF and PEG molecular weight itself play a determinant role in this phenomenon. Adding the reactive diluent i.e. NVP to the base macromer improves the chain mobility which enhances the reactivity of the components in turn, hence it is expected that the higher proportion of NVP, the greater the degree of conversion of the compositions. The present findings coincided with the expectations. FIG. 8 depicts degree of conversion for the crosslinked PEGF macromers containing diverse NVP contents.

To derive Mc from DMA, G′ was measured and static shear modulus (G) was then assumed by extrapolation of the rheogram to the zero frequency. The time scale for the indentation experiment was about 10 seconds in each measurement. Actually, we extrapolated oscillation graphs to 0.1 Hz. Moreover, we linearized these data by plotting G′ against log frequency. Extrapolated values of G′ were estimated to concentrations, respectively. Molecular weights between crosslinks (Mc) of the photocrosslinked gels were calculated using DMA, it means the G′ can be converted into crosslinking density (ρx) from rubber elasticity theory (eqs. (4, 5, 6)) [39, 40]:

$$\begin{matrix}{v}_{2 , s} = \frac{{V}_{d}}{{V}_{s}} = \frac{\frac{{W}_{d}}{{ρ}_{p}}}{\frac{{W}_{d}}{{ρ}_{p}} + [ \frac{{W}_{s} - {W}_{d}}{{ρ}_{o}} ]} & ( 4 ) \\ {G}^{′} = 2 {ρ}_{x} {RTv}_{2 , s}^{( 1 / 3 )} & ( 5 ) \\ {ρ}_{x} = \frac{1}{vMc} & ( 6 )\end{matrix}$$

here ρx is the crosslink density (moles of crosslinks per unit volume), R is the gas constant (8.314 JK-1Mol-1), T is the temperature (293 K), Mc is the average molecular weight between crosslinks, v2,s is the polymer fraction at equilibrium swelling and v is the partial specific volume of PEGF and Vd, Vs, Ws, Wd ρp and ρo are dry polymer volume (cm3), swollen polymer volume (cm3), swollen polymer weight (g), dry polymer weight (g), polymer density, solvent density (water) (g/cm3), respectively (Table 3).

TABLE 3

| | | Swelling | Sol |
|---|---|---|---|
| | % NVP | Ratio ± SD | Fraction ± SD |
| PEGF 0.4 kDa | 5 | 1.46 ± 0.10 | 25.92 ± 4.43 |
| | 8 | 1.09 ± 0.12 | 18.65 ± 2.91 |
| | 12 | 0.98 ± 0.09 | 15.22 ± 2.26 |
| PEGF 1 kDa | 5 | 0.61 ± 0.008 | 7.00 ± 0.41 |
| | 8 | 0.52 ± 0.01 | 7.20 ± 0.97 |
| | 12 | 0.52 ± 0.02 | 10.18 ± 1.27 |

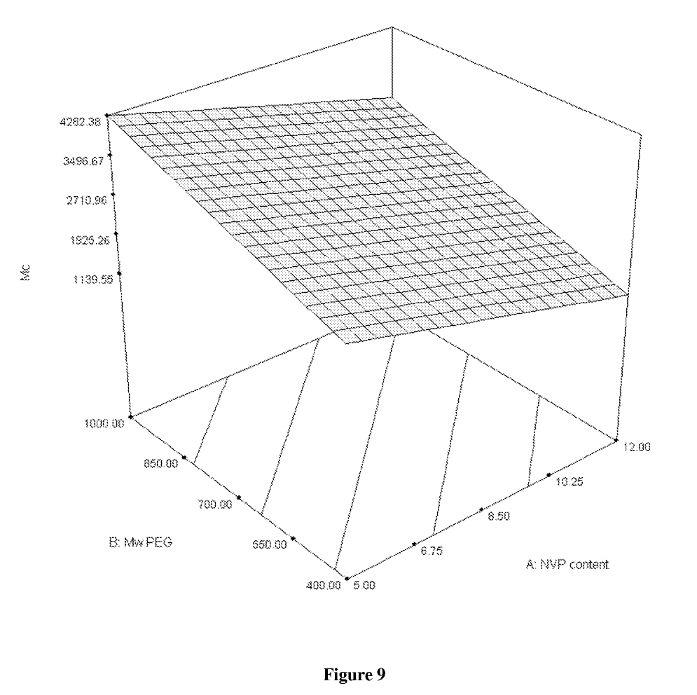

The elastic behavior of the samples prevails over its viscous behavior and the swollen gel exhibits mechanical rigidity. The increase in NVP content causes a decrease in Mc also rigidity of the PEGF gels. It seems that (FIG. 9), as the content of NVP is increased; the swelling ratio is decreased because the network tends to vary to a denser one.

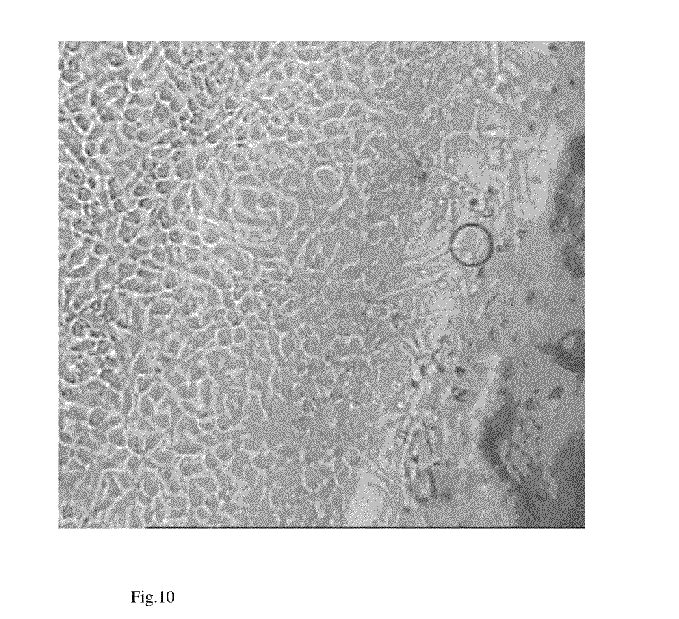

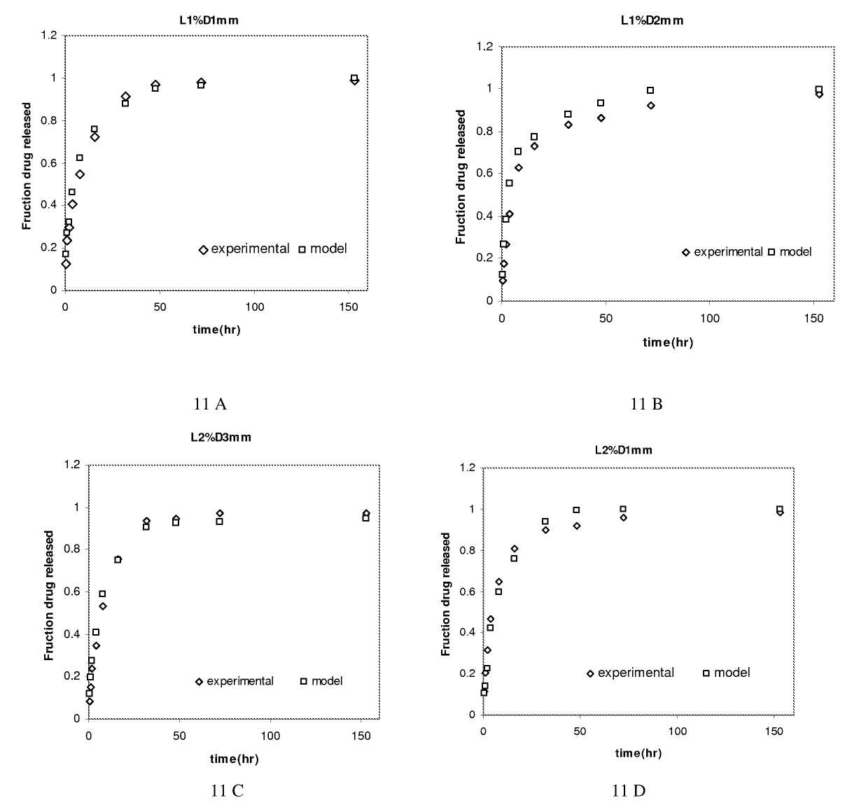

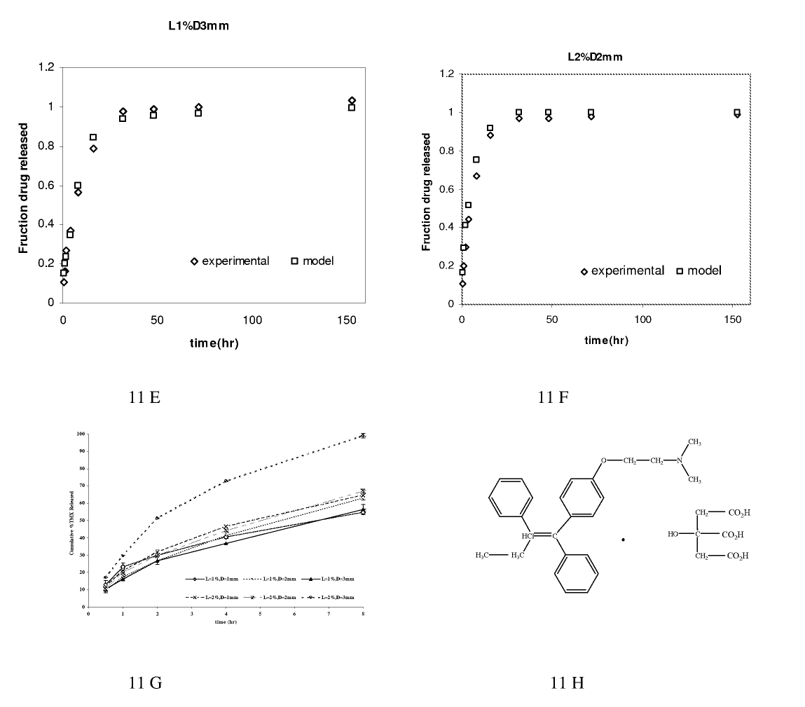

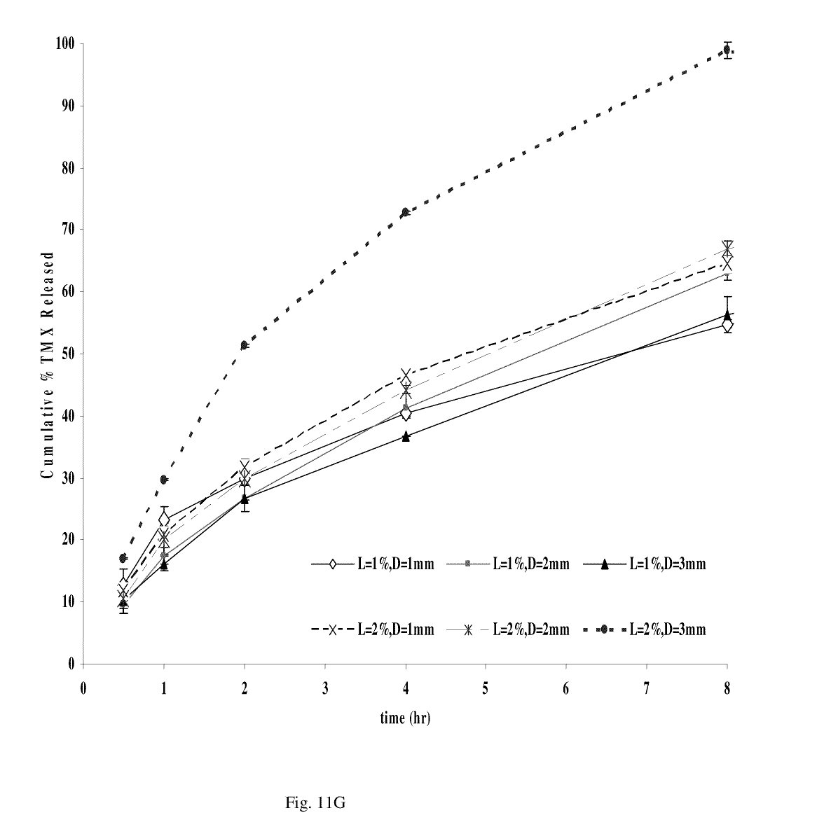

Cellular biocompatibility as determined by cell culture showed very good agreement with the results obtained from control samples. The solid photocrosslinked networks and their precursor uncured macromers were not toxic towards the cells at all. FIG. 10 depicts the morphology of L929 fibroblast cells cultured on photocrosslinked samples. As can be seen in this picture fibroblast cells could be considered completely flattened and well spread and elongating and expanding their filopedia.

As shown in FIG. 6, In vitro profiles release of tamoxifen citrate from PEGF 0.4K are seen as an example. The rapid burst effect is very much delayed when different loading technique is used for tamoxifen citrate. Based on the above finding, it observed that 1% drug loading and 2 mm shows optimum release characteristics. The release rate of tamoxifen citrate from PEGF 0.4K could be properly controlled for 8 h.

The macromers have potential to be used as in-situ forming injectable hydrogels systems. In vitro profiles release of tamoxifen citrate from PEGF 0.4K show the rapid burst effect is very much delayed when different loading technique is used for tamoxifen citrate. Appropriate variation in the proportions of drug and depth of matrix can lead to product with the desired controlled-release

### CONCLUSION

Unsaturated macromers were synthesized from diacids e.g. fumaryl chloride and diols e.g. polyethylene glycol in the presence of propylene glycol as a new proton scavenger and characterized as an injectable biomaterial. NVP was used as a crosslinking/reactive diluent agent to increase final double bond conversion and to reduce the composition viscosity hence, improving injectabiliy. The results showed that photocrosslinking was facilitated at higher NVP contents and shrinkage strain rate of PEGF/NVP mixtures followed the same pattern of polymerization reaction of multifunctional monomers showing auto-acceleration and auto-deceleration patterns. Total shrinkage strain of mixture was increased by increasing amount of NVP from 5% to 20% and increasing molecular weight. Mc was determined with ball indentation DMA which indicated an increasing crosslink density of networks and decreasing Mc upon an increase in the NVP content of the compositions. Cell biocompatibility evaluation of PEGF/NVP copolymers by general fibroblast cell culture showed that these materials are biocompatible and the solid photocrosslinked networks were not toxic towards the cells at all. Unsaturated macromers can be used as precursors to prepare polymeric networks and scaffolds with controlled hydrophilicity, swelling and mechanical properties for applications in drug release and tissue engineering. The macromers have potential to be used as in-situ forming injectable hydrogels systems.

The description of the embodiment set forth above is intended to be illustrative rather than exhaustive of the present invention. It should be appreciated that those of ordinary skill in the art may make certain modifications, additions or changes to the described embodiment without departing from the spirit and scope of this invention as claimed hereinafter.

We claim:

1. A method for controlled delivery of a predetermined amount of a drug wherein said method comprises;
Combining a macromer with a predetermined amount of a drug, and a plurality of initiators, and obtaining a first composition,
Applying a crosslinking procedure to said first composition, wherein said drug is unaffected by said crosslinking; and
Obtaining a second composition, wherein said second composition controls said delivery of said predetermined amount of said drug.

2. The method as claimed in claim 1, wherein said macromer comprises of unsaturated aliphatic polyesters, wherein said unsaturated aliphatic polyesters are a result of a polyesterification reaction catalyzed by a catalyst between a diol, wherein said diol comprises polyethyelene glycol, polycaprolactone diol, and polyhexamethylene carbonate diol and an acyl halide wherein said acyl halide comprises of fumaryl chloride and itaconyl chloride.

3. The method as claimed in claim 2, wherein said catalyst comprises of epoxy containing compounds wherein said compounds comprises propylene oxide, and 1,2-butylene oxide.

4. The method as claimed in claim 3, wherein said macromer is a white clear macromer.

5. The method as claimed in claim 4, wherein said macromer is crosslinked by visible light initiation, wherein said visible light initiation characterized with duration and intensity of said visible light irradiation.

6. The method as claimed in claim 4, wherein said macromer is crosslinked by thermal initiation, wherein said thermal initiation characterized with duration of crosslinking, temperature of crosslinking and concentration of said thermal initiation.

7. The method as claimed in claim 4, wherein said macromer is crosslinked by redox initiation, wherein said redox initiation characterized with duration of crosslinking, and concentration of said redox initiation.

8. The method as claimed in claim 4, wherein said controlled delivery of said predetermined amount of said drug is based on intensity and duration of said visible light irradiation.

9. The method as claimed in claim 5, wherein said controlled delivery of said predetermined amount of said drug is based on said duration of crosslinking, said temperature of crosslinking and said concentration of said thermal initiation.

10. The method as claimed in claim 6, wherein said controlled delivery of said predetermined amount of said drug is based on said duration of crosslinking, and said concentration of said redox initiation.

11. A composition for controlling duration and amount of delivery of a predetermined amount of a drug, wherein said composition comprises:
A first composition comprising a macromer, a predetermined amount of a drug, and a plurality of initiators, wherein said macromer is subject to a crosslinking procedure, thereby obtaining a second composition, wherein said second composition controls said duration and amount of delivery of said predetermined amount of said drug.

12. The composition as claimed in claim 11, wherein said macromer comprises of unsaturated aliphatic polyesters, wherein said unsaturated aliphatic polyesters are a result of a polyesterification reaction catalyzed by a catalyst between a diol, wherein said diol comprises polyethylene glycol, polycaprolactone diol, and polyhexamethylene carbonate diol and an acyl halide wherein said acyl halide comprises of fumaryl chloride and itaconyl chloride.

13. The composition as claimed in claim 12, wherein said catalyst comprises of epoxy containing compounds wherein said compounds comprises propylene oxide, and 1,2-butylene oxide.

14. The composition as claimed in claim 12, wherein said macromer is a white clear macromer.

15. The composition as claimed in claim 12, wherein said macromer is crosslinked by visible light initiation, wherein said visible light initiation characterized with duration and intensity of said visible light irradiation.

16. The composition as claimed in claim 12, wherein said macromer is crosslinked by thermal initiation, wherein said thermal initiation characterized with duration of crosslinking, temperature of crosslinking and concentration of said thermal initiation.

17. The composition as claimed in claim 12, wherein said macromer is crosslinked by redox initiation, wherein said redox initiation characterized with duration of crosslinking, and concentration of said redox initiation.

18. The composition as claimed in claim 12, wherein said controlled delivery of said predetermined amount of said drug is based on intensity and duration of said visible light irradiation.

19. The composition as claimed in claim 12, wherein said controlled delivery of said predetermined amount of said drug is based on said duration of crosslinking, said temperature of crosslinking and said concentration of said thermal initiation.

20. The composition as claimed in claim 12, wherein said controlled delivery of said predetermined amount of said drug is based on said duration of crosslinking, and said concentration of said redox initiation.
